(19) United States
(12) Patent Application Publication
(10) Pub. No.: US 2026/0069890 A1
(43) Pub. Date: Mar. 12, 2026

(54) SYSTEM AND METHOD FOR REAL-TIME IMAGE REGISTRATION DURING RADIOTHERAPY USING DEEP LEARNING

(71) Applicant: Mary Hitchcock Memorial Hospital, for itself and on behalf of Dartmouth-Hitchcock Clinic, Lebanon, NH (US)

(72) Inventors: Brady N. Hunt, Lebanon, NH (US); Rongxiao Zhang, West Lebanon, NH (US)

(21) Appl. No.: 18/869,219

(22) PCT Filed: May 26, 2023
(86) PCT No.: PCT/US2023/023660

§ 371 Date: Nov. 25, 2024

Related U.S. Application Data
(60) Provisional application No. 63/346,299, filed on May 26, 2022.

(51) Int. Cl. A61N 5/10; G06T 7/33

(52) U.S. Cl. CPC A61N 5/1067; A61N 5/1049; G06T 7/33; A61N 2005/1055; G06T 2207/10088; G06T 2207/20081; G06T 2207/20084

(57) ABSTRACT

This invention provides a deep learning (DL) model for fast deformable image registration using 2D sagittal cine MRI acquired during radiation therapy. A DL model for fast deformable image registration is trained using cine MRI scans acquired during MR-Linac treatments of thoracic and abdominal tumors. The model uses a pair of cine MRI images as inputs and outputs a dense motion vector field (MVF) which aligns the images. The trained model is applied to predict frame by frame motion from cine MRIs in which both cardiac and respiratory motion are visible. The number of respirations and heart beats is automatically extracted by performing peak detection on high-frequency and low-frequency components of the MVF displacements corresponding to the chest wall and cardiac regions.

### FIELD OF THE INVENTION

This invention relates to medical imaging, and more particularly to registration of images during runtime to guide radiotherapy relative to selected regions of a patient's anatomy.

### BACKGROUND OF THE INVENTION

Management of patient motion during radiation therapy is a long-standing clinical challenge, particularly in treating abdominal and thoracic tumors. This has led to the advent of real-time MRI-guided radiation therapy (MRgRT) systems, which continuously acquire cine MRI during treatment to monitor internal motion at the treatment site. Currently, two of the commercially available systems supporting real-time MRgRT are the MRIdian, available from View Ray of Oakwood Village, OH and the Unity, available from Elekra Solutions AB of Sweden, which support 2D cine acquisition rates of approximately 8 and 5 frames per second (fps), respectively. The high temporal resolution MRI data available through these systems represents a rich data source with which to study intrafraction patient motion and develop improved methods to account for patient motion during radiation therapy. However, to date, only a small number of studies have analyzed cine MRI datasets acquired from MR-LINACs. Recent work highlights a fundamental trade-off in MRgRT between achieving low latency for real-time monitoring while preserving adequate image quality for downstream analysis (target tracking and fraction review).

In 2015, an initial study investigated automated tracking using cine MRI from the first generation View Ray system. See Mazur, T. R., Fischer-Valuck, B. W., Wang, Y., Yang, D., Mutic, S. & Li, H. H. SIFT-based dense pixel tracking on 0.35 T cine-MR images acquired during image-guided radiation therapy with application to gating optimization: SIFT-based dense pixel tracking of 0.35 T cine-MRI. Med. Phys. 43, 279-293 (2015). The chosen operational algorithm accounted for deformable motion by estimating a dense displacement field using key point matches from pixels sampled at regular intervals throughout the target region of interest. The analysis therein included cine MRI acquired at 4 fps from a single treatment fraction of 19 patients with abdominal and thoracic tumors. It characterized the magnitude of tumor target respiratory motion in both superior-inferior (SI) and anterior-posterior (AP) directions, observing that SI motion tended to be larger in magnitude and ranged from 0.6 to 3.0 cm. The Mazur method is able to achieve a mean Dice similarity coefficient of 0.92 for automated tracking contours compared to manually drawn contours across all patients in their cohort with a computation time ˜250 ms per registration.

Two more recent studies investigated radial sampling in conjunction with deep learning (DL)-based methods to achieve motion tracking with even lower latency. One study acquired cine MRIs using a 1.5 Tesla scanner from a cohort of 135 patients with abdominal tumors. See Terpstra, M. L., Maspero, M., d'Agata, F., Stemkens, B., Intven, M. P. W., Lagendijk, J. J. W., van den Berg, C. A. T. & Tijssen, R. H. N. Deep learning-based image reconstruction and motion estimation from undersampled radial k-space for real-time MRI-guided radiotherapy. Phys. Med. Biol. 65, 155015 (2020). The cine MRI scans are not taken using an MR-LINAC as part of radiation therapy, but are performed prior to treatment as part of simulation and treatment planning. This study utilized an early DL model for estimating motion vector fields (MVFs) (a spatial pyramid network) and demonstrated that this model could outperform conventional registration methods, but only in the cases of highly under sampled k-space data (conventional methods outperformed on fully sampled data). In principle, it is recognized that combining the DL model with conventional radial reconstruction methods could reduce the latency of real-time monitoring with cine to approximately 60 ms (16 fps): however, this sparse cine MRI reconstruction exhibited radial artifacts, potentially making it difficult for clinicians to use for fraction review. Another study demonstrated a similar finding using clinical cine MRI data from the View Ray system. See Friedrich, F., Hörner-Rieber, J., Renkamp, C. K., Klüter, S., Bachert, P., Ladd, M. E. & Knowles, B. R. Stability of conventional and machine learning-based tumor auto-segmentation techniques using undersampled dynamic radial bSSFP acquisitions on a 0.35 T hybrid MR-linac system. Med. Phys. 48, 587-596 (2021). It utilized radial k-space sampling in conjunction with a U-Net to generate the tracking contours with latency as low as 94 ms (10.6 fps).

A recent study investigated the potential for deep learning to improve the spatial resolution of the low-field strength cine MRIs, which could in theory enable shorter scan times while preserving high image quality 9.10. In their initial study, they trained a generative adversarial network (GAN) for low-resolution to high-resolution MRI conversion using phantom/volunteer cine MRI scans acquired with the first generation ViewRay system. See Chun, J., Zhang, H., Gach, H. M., Olberg, S., Mazur, T., Green, O., Kim, T., Kim, H., Kim, J. S., Mutic, S. & Park, J. C. MRI super-resolution reconstruction for MRI-guided adaptive radiotherapy using cascaded deep learning: In the presence of limited training data and unknown translation model. Med. Phys. 46, 4148-4164 (2019). The original scans are acquired using longer scan times and at 256×256 pixels with a spatial resolution of 1.5 mm. These scans are then down sampled to 64×64 pixels (6.0 mm resolution) and used as inputs to train the network to predict the paired high-resolution scan. Their results indicated that the network improved spatial resolution by ˜4× and could potentially be utilized with faster acquisition schemes while preserving image quality. Follow up work then demonstrated that the previously trained model could also improve cine image quality from cine acquired using the second-generation ViewRay system (3.5 mm resolution and 4 fps). See Chun, J., Lewis, B., Ji, Z., Shin, J.-I., Park, J. C., Kim. J. S. & Kim, T. Evaluation of super-resolution on 50 pancreatic cancer patients with real-time cine MRI from 0.35 T MRgRT. Biomed. Phys. Eng. Express 7, 055020 (2021). However, such research has not yet assessed if their DL-enhanced cine MRI could improve downstream image registration and motion tracking.

In other domains of radiological imaging, DL-based image registration methods have grown rapidly in recent years. See Haskins. G., Kruger. U. & Yan. P. Deep learning in medical image registration: a survey. Machine Vision and Applications 31, 8 (2020); and Fu, Y., Lei, Y., Wang, T., Curran, W. J., Liu, T. & Yang, X. Deep learning in medical image registration: a review. Phys. Med. Biol. 65, 20TR01 (2020). One of the key developments has been the application of self-supervised learning (SSL), which overcomes the need for manual annotation of every training sample (i.e. supervised learning). This implementation is highly beneficial in the case of video rate datasets, such as cine MRI, as such are very time intensive to manually annotate. More generally, it is highly desirable to provide a system and method that registers medical imagery in real time with higher accuracy and lower latency to allow for guidance of treatment in a reliable manner.

### SUMMARY OF THE INVENTION

This invention overcomes disadvantages of the prior art by providing a deep learning (DL) model for fast deformable image registration using Cine MRI (or by way of particular example, 2D sagittal cine MRI) acquired during radiation therapy. A DL model for fast deformable image registration is trained using cine MRI scans acquired during MR-Linac treatments of thoracic and abdominal tumors. The model uses a pair of cine MRI images as inputs and outputs a dense motion vector field (MVF) which aligns the images. In one example, which can be applied to cardiac monitoring, The trained model is then applied to predict frame by frame motion from cine MRIs in which both cardiac and respiratory motion are visible. The number of respirations and heart beats are automatically extracted by performing peak detection on high-frequency and low-frequency components of the MVF displacements corresponding to the chest wall and cardiac regions. The number of respirations and heart beats are also counted manually for comparison. Frequency analysis of MVF time series is performed to estimate the heart rate (HR) in beats per minute of each patient across multiple fractions.

In an illustrative embodiment, a system and method for fast deformable image registration using 2D cine MRI image frames, acquired during radiation therapy, is provided. A source of cine MRI images is received in approximate real-time from a patient undergoing MRI-guided radiation therapy (MRgRT). A processor receives the images and operates a trained deep learning (DL) process that (a) employs a reference image (R) to a moving image as inputs, and outputs a dense motion vector field (MVF) that aligns the images and (b) predicts frame by frame motion from images. A guidance process that controls therapy beam operation based upon the prediction. Illustratively, both cardiac and respiratory motion can be visible in the images. The DL process can be adapted to extract, from the images, a predetermined number of hierarchical feature maps at multiple spatial resolutions which are reconstructed into the MVF with spatial resolution equal to the inputs. The MVF can be applied by shifting the pixels in the images by associated motion vectors to generate a transformed image (T), and the transformed image can be generated via a spatial transform. Illustratively, the DL process can define a plurality of network layers and at least one of the network layers is based on a U-Net architecture of a convolutional neural network (CNN). A loss function can ne provided with a hyperparameter (λ) defined as:

$${Loss}_{total} = {Loss}_{dissimilarity} + λ {Loss}_{gradients}$$

where, Loss_{dissimilarity} computes the post-registration mean square error between T and R and Loss_{gradients} computes the magnitude of spatial gradients in the MVF. The network can be provided with network layers in the following order, for each successive layer, of spatial dimensions: 1, ½, ¼, ⅛, 1/16, ⅛, ¼, ½, 1. Additionally, the loss function can be defined by:

$${Loss}_{dissimilarity} = \frac{1}{mn} {\sum }_{i = 1}^{m} {\sum }_{j = 1}^{n} {( {T}_{i , j} - {R}_{i , j} )}^{2}$$

$${Loss}_{gradients} = \frac{1}{mn} {\sum }_{i = 1}^{m} {\sum }_{j = 1}^{n} {‖ ∇ ( {ϕ}_{i , j} ) ‖}^{2}$$

where m and n represent the height and width of images. In a further embodiment, the system and method can further include a combination process(or) that coordinates an EKG signal derived from the patient with the cine MRI images so as to provide data related to cardiac and respiratory motion to the DL process. The EKG signal can be derived from one or two legs of the patient so as to avoid interference from chest-based electrodes.

### BRIEF DESCRIPTION OF THE DRAWINGS

The invention description below refers to the accompanying drawings, of which:

FIG. 1 is a schematic diagram showing an overview of a MRI-guided radiation therapy (MRgRT) system and associated image processing arrangement employing a deep learning (DL) model to track and guide a treatment beam relative to a patient's region of interest;

FIG. 2 is graph showing temporal analysis of cine MRI frames in treatment delivery video including beam on/off, tracking confidence, and frame-to-frame root mean square error (RMSE) for the arrangement of FIG. 1;

FIG. 3 is a diagram of images and resulting deformable registration using three different cine MRI frames during inspiration for the arrangement of FIG. 1;

FIG. 3A is a flow diagram for a generalized flow diagram showing a retrospective analysis performance of the DL model according to the illustrative embodiment;

FIG. 3B is a diagram showing error and smoothness between an exemplary reference image and transformed image;

FIG. 4 is a flow diagram of DL model inputs and model architecture according to an illustrative embodiment;

FIG. 5 is a pair of exemplary images processed by DL model showing) pre-registration and post-registration;

FIG. 6 is a table of exemplary patient characteristics, cine tracking information, and machine learning fold assignments for use in the DL model and analysis herein;

FIGS. 7 and 8 are, respectively, cine frames for each exemplary patient imaged with arrangement of FIG. 1 during beam on and beam off time points;

FIGS. 9 and 10 are, respectively, time series extracted and exemplary image frames from MR-Linac delivery tracking videos from two patients undergoing MRgRT for lung tumors, including a time series of mean frame intensity and tracking confidence for a first patient and a second patient exhibiting substantial variation in tracking confidence;

FIGS. 11 and 12 are, respectively, exemplary image frames for beam on and beam off for the first patient of FIG. 9 and the second patient of FIG. 10;

FIG. 13 is a set of images comprising a reference image, moving image and stereo color visualization of image misalignment by way of example;

FIGS. 14 and 15 are, respectively, graphs showing mean registration error (RMSE) and fraction of folding pixels (negative Jacobian determinant) on all test set images employing images such as FIG. 13, and based upon the DL model versus other conventional registration methods;

FIG. 16 is a visualization of deformation fields and residual error for various configurations of the DL registration model smoothing hyperparameter (λ), including a color plot of Jacobian determinant on example images for each λ, grid plot of predicted deformation for each λ, and resulting images from applying the DL model registration to the reference image;

FIG. 17 is a graph showing a comparison of registration error across all patients and treatment sites relative to a mean registration error for all cine sequences for each patient;

FIG. 18 is a box and whisker distribution plot of registration error for all cine sequences stratified by treatment site (liver, lung, and pancreas) as well as for the entire dataset;

FIG. 19 is a diagram and images of registration error and deformation smoothness metrics for an exemplary cine sequence for each of the patients analyzed using the illustrative embodiment, and applying the DL model versus conventional techniques; and

FIG. 20 is an exemplary operational setup for an MR-Linac arrangement according to an illustrative embodiment including used of an EKG monitoring arrangement.

### DETAILED DESCRIPTION

### I. System and Terms

### A. Definitions

The following terms are defined herein:
AP: Anterior-posterior
DIBH: Deep inspiration breath hold
DL: Deep learning
FB: Free breathing
FPS: Frames per second
MRgRT: MRI-guided radiation therapy
MR-LINAC: MRI-guided linear accelerator
MVF: Motion vector field
RMSE: Root mean square error
SBRT: Stereotactic-body radiation therapy
SSL: Self-supervised learning
SI: Superior-inferior

### B. Data Acquisition

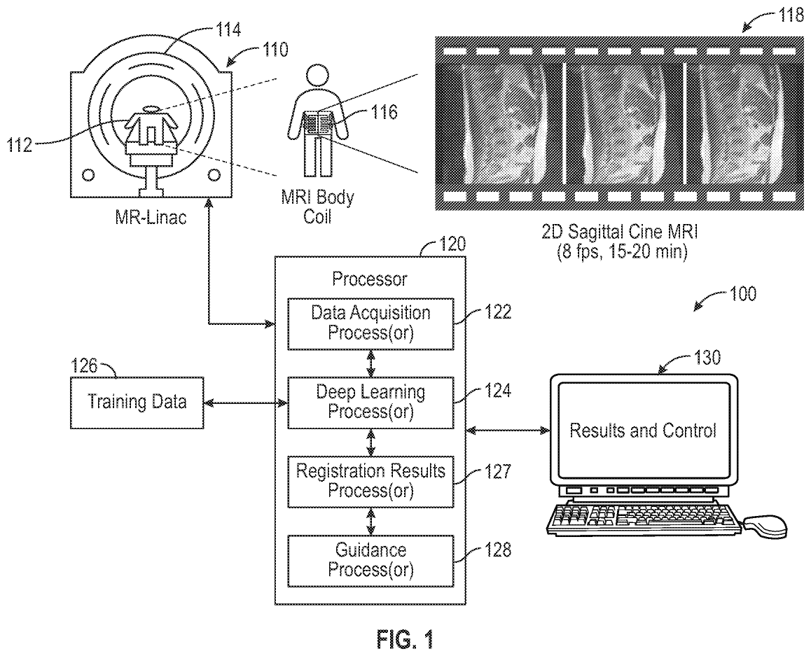

Reference is made to FIG. 1, which shows a schematic diagram of an imaging and processor arrangement 100 according to an embodiment. A MR-Linac cine acquisition imaging system 110 is shown with an associated image processing assembly 120. The MR-Linac 110 is shown with an exemplary patient 112 imaged using a field generated by the device magnetic ring 114 interoperating with a patient-mounted body coil 116. Frames 118 of cine MRI are thereby acquired at (e.g.) 8 fps, and a typical treatment duration can be 15-20 minutes. The processor 120 can be instantiated in a purpose-built or conventional computing device. It can be provided as a single device for example a general purpose computer architecture (e.g. PC, server, laptop, tablet, cloud computing arrangement, etc.) 130 for controlling operation of the arrangement 100 and/or displaying and reporting results: or processing functions can be distributed among different devices—for example a general purpose computer and a graphical processing unit (GPU).

The processor 200 includes various functional processes/ors and/or operation modules that can be instantiated as hardware, software, including a computer-readable medium of non-transitory program instructions, or a combination of hardware and software. The depicted names and organization of such processes/ors is exemplary and can differ as appropriate in a manner known to those of skill in the art. As shown, a data acquisition process(or) 122 receives cine image data from the system 110 and extracts appropriate features. These features are processed by a deep learning (DL) process(or) 124, that can be based upon a convolutional neural network (CNN) as described below. The CNN uses training results to identify features of interest (i.e. tumorous tissue to be targeted by the system's treatment beam). The CNN interoperates with a registration process(or) 127 that provides registration results on features of interest, requiring treatment by the system's beam. These results are used by a guidance process(or) 128 to thereby guide beam operation and timing so as to help ensure the beam is localized in the treatment region.

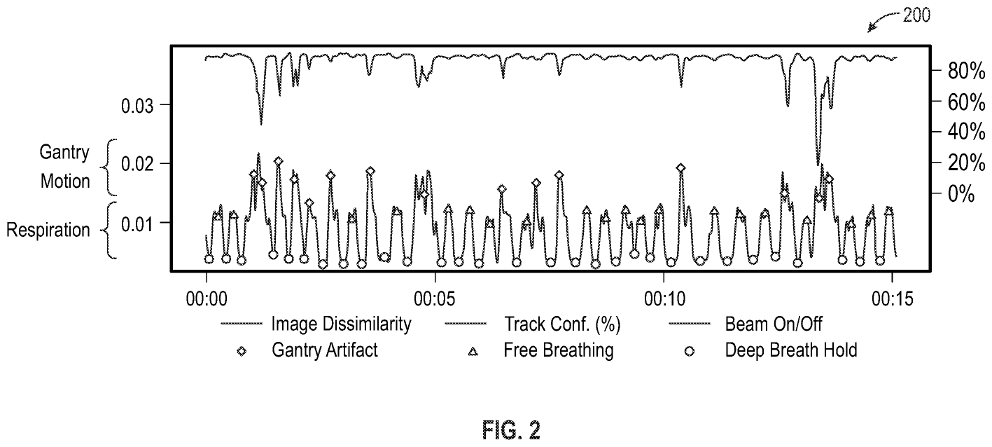

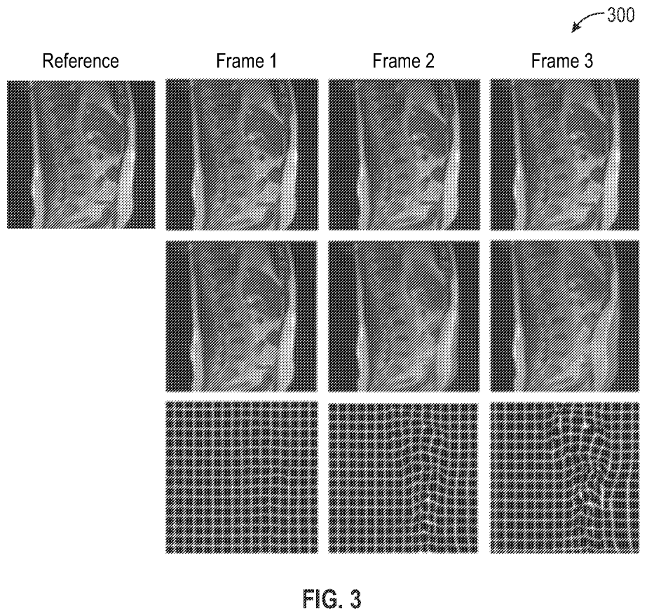

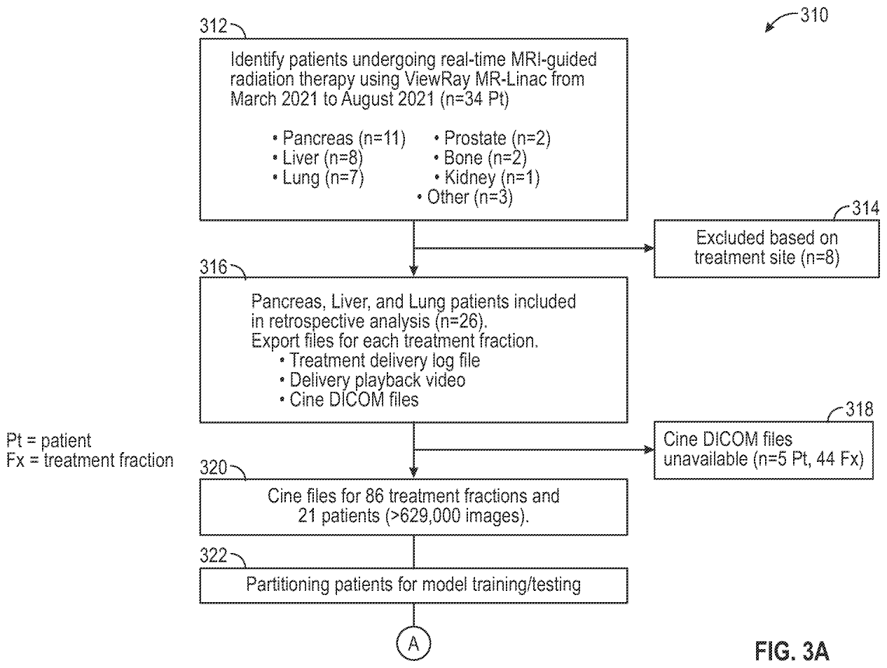

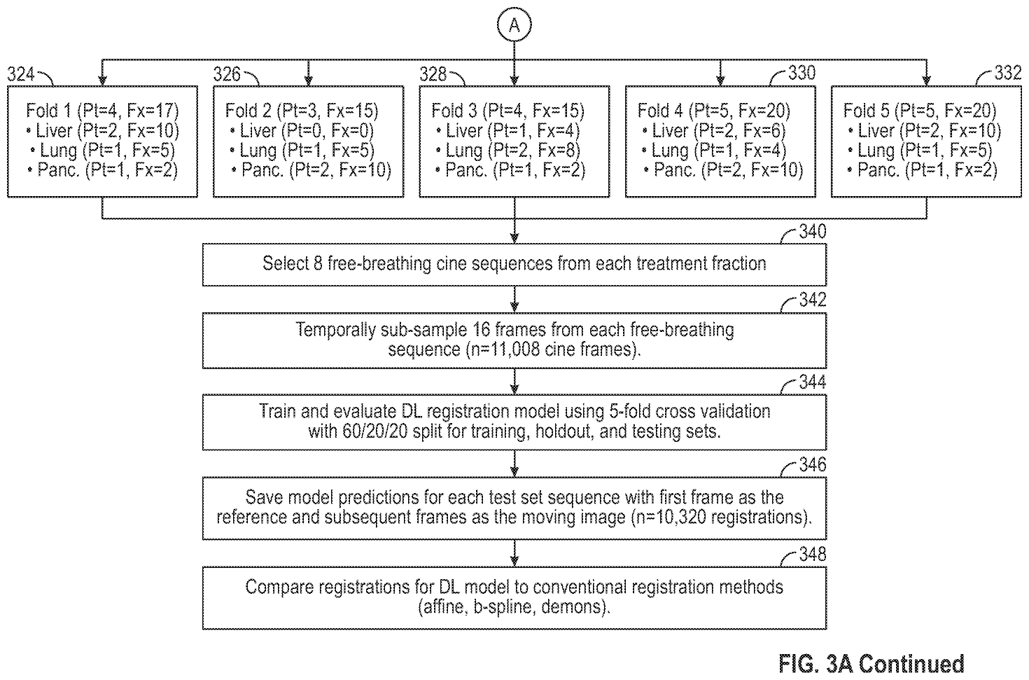

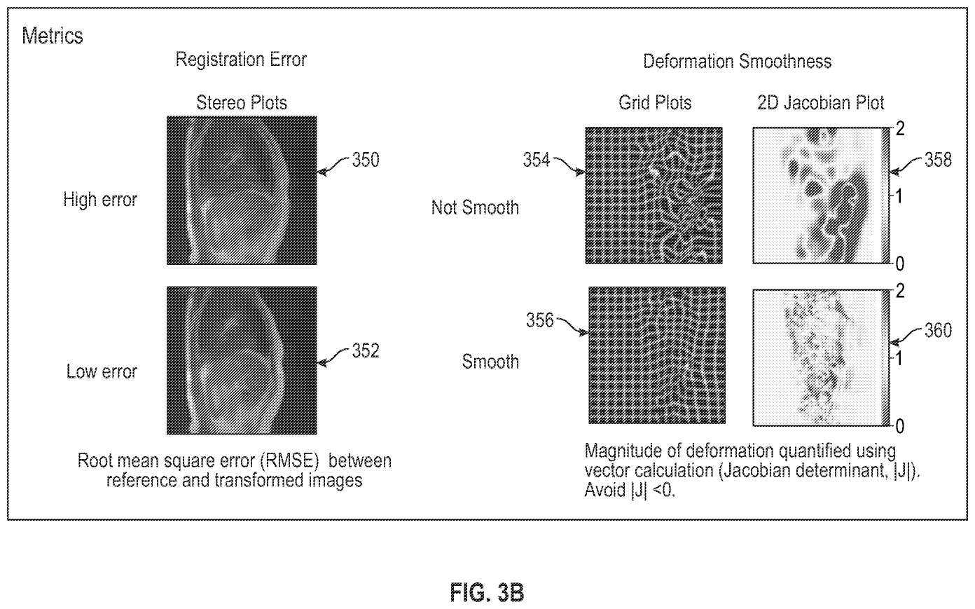

FIG. 2 is a graph 200 showing temporal analysis of cine MRI frames in treatment delivery video including beam on/off, tracking confidence, and frame-to-frame RMSE. Peak detection is performed on the timeseries to subsample cine frames between deep inspiration breath hold and free breathing time points. As described further below, FIG. 3, shows an example of deformable registration using three different cine MRI frames (Frame 1-Frame 3) during inspiration. The exemplary MR-LINAC system 110 utilized in this example is the View Ray MRIdian. During treatment, a sagittal 2D cine MRI is continuously acquired by the low-field MRI scanner onboard the View Ray system (e.g., Siemens Avanto, 0.35 Tesla) using a TRUFI (True Fast Imaging with steady-state-free precession) pulse sequence with radially subsampled reconstruction. The resultant cine images 118 have 350 mm field-of-view and a native resolution of 144×144 pixels (2.43 mm/pixel) at 8 fps. Note that the parameters provided herein should be taken as exemplary of a particular setup and MR-Linac arrangement (e.g. View Ray), and not to otherwise limit the applicability of the principles herein to other models/arrangements of MR-Linac that should be clear to those of skill. In the depicted exemplary image frames, patients are performing deep inspiration breath holds during the treatment course to engage the gating system via automated target tracking: thus, delivery cine videos primarily consist of alternating sequences of breath holding and free breathing, with sporadic instances of gantry motion artifacts when the beam delivery angle is changing as shown by certain graph peaks in FIG. 2. Delivery cine images are herein accessed in two formats: (a) video file exported from the View Ray treatment planning software showing tracking contours and confidence metrics overlaid on video, and (b) DICOM files saved via the Siemens Avanto console for quality assurance review. Additionally, ViewRay machine log files documenting the date of radiotherapy, tracking algorithm parameters, couch position, and tracking slice position for each treatment fraction are accessed.

### C. Data Partitioning and Temporal Sampling

FIG. 3A shows an overall flow diagram 310 of a retrospective analysis study design used to develop the system and method herein, with specific examples. This analysis is referred to variously in following description. The procedure 310, in step 312 identifies patients undergoing real-time MRgRT using e.g. the View Ray MR-Linac system with various conditions, including those involving the pancreas, lung, liver, prostate, bone, kidney and other. Some patients are excluded based on treatment site (step 314). For pancreas and liver patients treatment delivery log files, delivery playback video and cone DICOM files are exported for each treatment fraction (step 316). Certain cine DICOM files are shown as unavailable in this step (block 318). In the example, 86 cine files relative to 21 patients >629,000 images) are available to the procedure 310 (block 320). As shown in step 322, in order to avoid overfitting the DL model to patient-specific features, patients are initially stratified by treatment site and total number of fractions and then randomly assigned to one of five partitions 324, 326, 328, 330 and 332. These patient assignments are utilized in a 60/20/20 cross-validation scheme where 60% of patients are used during training to update model parameters, 20% withheld from training but used to monitor model performance on unseen data, and 20% withheld as an independent test set for evaluating final model performance (see step 344). Additionally, because each cine MRI has a variable duration, a fixed number of frames from each fraction are temporally sampled through semi-automated review as follows. For each treatment fraction analyzed, the View Ray delivery tracking video is processed using a Python script to extract tracking confidence, beam on/off status, mean frame intensity, and frame-to-frame root mean square error (RMSE) to generate a timeseries plot (200 in FIG. 2) for each video. Frames affected by gantry motion artifacts exhibit large intensity deviations and can be excluded using a threshold on the mean frame intensity values (±20% from the median frame intensity across the entire video). Frames within 2 seconds (16 frames) of these large intensity deviations are excluded. Timeseries plots are reviewed side-by-side with the video, and parameter thresholds are set to label time points corresponding to deep inspiration breath holds (DIBH, high tracking confidence, low frame-to-frame RMSE, and beam on) and free breathing (FB, higher frame-to-frame RMSE and lower tracking confidence). Frames from each cine are then sampled by identifying 8 individual time segments consisting of a DIBH, following by FB, and then a second DIBH (step 340) with no gantry artifacts in between. Sixteen frames (step 342) are sampled at regular intervals from each time segment, resulting in 128 cine frames from each treatment fraction. Prior to registration analyses, cine image data are rescaled to range from 0 to 1 using the 99.9th percentile intensity of cine for each treatment fraction and saved as 32-bit floating point arrays (step 346). All subsequent analyses can be based on the subsampled and normalized cine frames. As described below, the procedure 310 then seeks to compare registrations from the DL model to other conventional registration techniques, such as affine, b-spline and demons (step 348).

FIG. 3B shows relative metrics by way of illustration of the concepts herein related to differences between accurate and inaccurate registration, and how such is depicted in various graphics. Hence, stereo images 350 and 352 show respective high registration error and low registration error, which is particularly characterized as RSME between the reference and transformed images. Likewise, deformation smoothness, which is related to registration accuracy, and quantified using a vector calculation (e.g. a Jacobian determinant) is shown in not smooth and smooth grid plots 354 and 356, respectively. 2D Jacobian plots 358 and 360 are, likewise, shown.

### D. Deep Learning Model Optimization

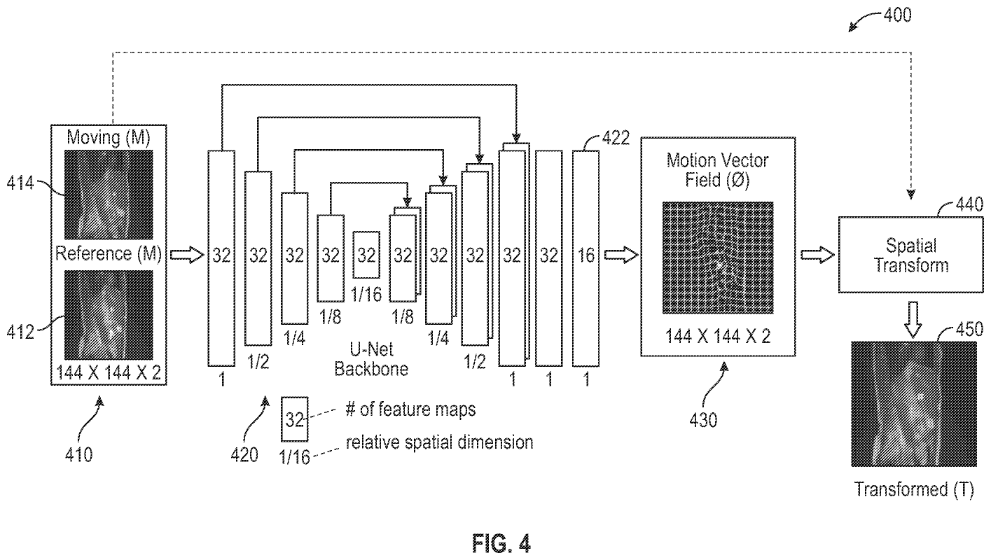

FIG. 4 shows an overview of the proposed DL model 400 for fast deformable registration according to the exemplary system and method herein, characterized by a flow diagram of model inputs and model architecture. A pair of images (referred as the reference (412) and moving (414) image) 410 are input into a convolutional neural network 420. The network outputs a motion vector field 430, which is applied to align the moving image 414 to the reference image 412, illustrates the DL model used for deformable registration of cine frames. The DL model can be adapted from an open-source, SSL model called VoxelMorph. See, by way of useful background Balakrishnan, G., Zhao, A., Sabuncu, M. R., Guttag, J. & Dalca, A. V. VoxelMorph: A Learning Framework for Deformable Medical Image Registration. IEEE Trans. Med. Imaging 38, 1788-1800 (2019). More particularly, the illustrative system and method employs the moving image 414 (also denoted as M in FIG. 4) and the reference image 412 (also denoted as R in FIG. 4), concatenated as a 144×144×2 numerical array input to the network 420. The model 400 extracts hierarchical feature maps (each denoted by the number of feature maps (e.g. 32) and the relative spatial dimension (e.g. in the following order: 1, ½, ¼, ⅛, 1/16, ⅛, ¼, ½, 1, 1, 1)) at multiple spatial resolutions which are reconstructed into MVF (φ) with spatial resolution equal to the inputs. The final layer 422 input to the MVF 430 contains 16 feature maps. The motion vector field (MVF) 430 is then applied by shifting the pixels in M by their associated motion vectors to generate (via spatial transform 440) a transformed image 450 (also denoted as T). The majority of the network layers are based on the U-Net architecture, which is widely used in medical image segmentation tasks. See, by way of useful background information, Ronneberger, O., Fischer. P. & Brox, T. U-Net: Convolutional Networks for Biomedical Image Segmentation, in Medical Image Computing and Computer-Assisted Intervention-MICCAI 2015 (eds. Navab, N., Hornegger, J., Wells, W. M. & Frangi, A. F.) vol. 9351 234-241 (Springer International Publishing, 2015). The primary differences are the addition of a spatial transform layer and an appropriate loss function for learning the image registration task. More specifically, the loss function of the model involves two terms which represent the fundamental trade-off in image registration: finding a transform which minimizes registration error while simultaneously being constrained to be physically realistic. The relative importance of these aspects in the loss function is balanced by means of a hyperparameter (λ):

$${Loss}_{total} = {Loss}_{dissimilarity} + λ {Loss}_{gradients} \qquad ( 1 )$$

where, Loss_{dissimilarity} computes the post-registration mean square error between T and R and Loss_{gradients} computes the magnitude of spatial gradients in the predicted MVF. These two loss terms are computed as follows:

$${Loss}_{dissimilarity} = \frac{1}{mn} {\sum }_{i = 1}^{m} {\sum }_{j = 1}^{n} {( {T}_{i , j} - {R}_{i , j} )}^{2} \qquad ( 2 )$$

$${Loss}_{gradients} = \frac{1}{mn} {\sum }_{i = 1}^{m} {\sum }_{j = 1}^{n} {‖ ∇ ( {ϕ}_{i , j} ) ‖}^{2} \qquad ( 3 )$$

where m and n represent the height and width of images.

It should be clear to those of skill that the particular deep learning model used in the illustrative embodiment is exemplary of a wide range of CNN architectures and other AI approaches that can be trained to recognize and track features in 2D scan images.

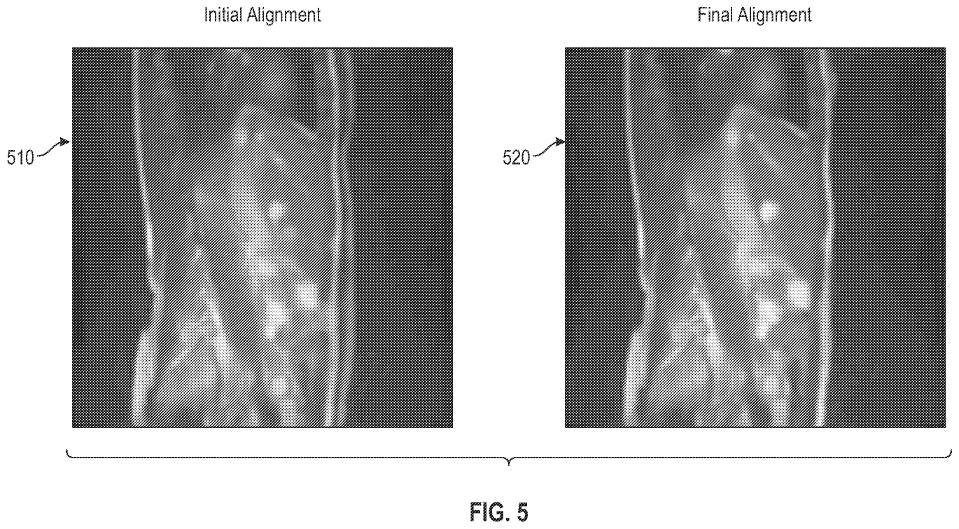

Optimization of the DL model is performed for all five data splits using (e.g.) custom Python scripts and the TensorFlow library. See Martin Abadi, Ashish Agarwal, Paul Barham, Eugene Brevdo, Zhifeng Chen, Craig Citro. Greg S. Corrado, Andy Davis, Jeffrey Dean, Matthieu Devin, Sanjay Ghemawat, Ian Goodfellow. Andrew Harp, Geoffrey Irving, Michael Isard, Jia, Y., Rafal Jozefowicz, Lukasz Kaiser, Manjunath Kudlur, Josh Levenberg, Dandelion Mane, Rajat Monga, Sherry Moore, Derek Murray, Chris Olah, Mike Schuster, Jonathon Shlens, Benoit Steiner, Ilya Sutskever, Kunal Talwar, Paul Tucker, Vincent Vanhoucke. Vijay Vasudevan, Fernanda Viegas, Oriol Vinyals, Pete Warden, Martin Wattenberg, Martin Wicke, Yuan Yu, & Xiaoqiang Zheng. TensorFlow: Large-Scale Machine Learning on Heterogeneous Systems. (2015). The exemplary computing arrangement employed to validate the system and method is configured with dual CPUs (Xeon Gold 5218), 256 GB of RAM, and two NVidia 2080 Ti graphics cards. Values for λ ranging over eight orders of magnitude (from 1×10⁻⁸ up to 1) have been tested to evaluate the impact of the gradient regularization on registration error. For each λ value, training is run for 50k steps with a batch size of 8 images using the Adam optimizer configured with a learning rate constant of 0.001 and decay constants of 0.9 and 0.999 for first and second moment estimates, respectively. Holdout training data is evaluated every 100 steps, and final model parameters is saved for the best performing model. Once final model parameters are selected, the DL model is used to perform image registration on all test set images, saving both transformed images and MVFs for subsequent analyses. As shown in FIG. 5, using the DL model above, a pre-registration image 510 is aligned into the depicted post-registration image 520.

### II. Comparison of DL Model and Conventional Techniques

### A. Conventional Image Registration

As described in step 348 of FIG. 3A, to evaluate DL model registration performance, three conventional registration methods are also utilized: affine, b-spline, and demons. Software scripts to process and register cine MRI sequences using conventional methods can be implemented using an open-source Python-based library of the Insight Segmentation and Registration Toolkit (ITK). See Yaniv, Z., Lowekamp. B. C., Johnson, H. J. & Beare, R. SimpleITK Image-Analysis Notebooks: a Collaborative Environment for Education and Reproducible Research. J Digit Imaging 31, 290-303 (2018). For both affine and b-spline approaches, the general configuration is typically the same. Mean square error (MSE) can be set as the optimization metric and a L-BFGS optimizer is run for a maximum of 50 iterations with early stopping if the gradient tolerance reaches 1×10⁶ or if the relative reduction in MSE is less than 1×10⁻⁸. In the case of affine, all six degrees of freedom are fit. For the b-spline transform, a 19×19 mm mesh grid with 832 fit parameters can be used. For demons registration, the fast symmetric forces variant of the algorithm using a finite difference solver can be run for 200 iterations without early stopping criteria. See, by way of useful background, Vercauteren, T., Pennec, X., Perchant, A. & Ayache, N. Diffeomorphic demons: Efficient non-parametric image registration. NeuroImage 45, S61-S72 (2009). In this example, all computations are performed on the same computing system as the DL model optimization, with multi-threading enabled to leverage all CPU cores. For each registration, the computational time required, number of model iterations, transformed images and MVF of the transform can be saved for subsequent quantitative comparisons with the DL model.

### B. Comparison Metrics and Statistical Analyses

Performance has been compared across all registration methods using 128 cine frames sampled from 8 different time points in each treatment fraction analyzed (step 348 in FIG. 3A). These frames are used to construct 120 registration pairs per treatment fraction by using the first frame from each sequence as a reference frame and the subsequent frames as moving images. Performance metrics are three-fold: (a) registration error, (b) MVF smoothness (both spatial and temporal), and (c) average computation time. Registration error is quantified using RMSE between the transformed and reference images. Spatial MVF smoothness is quantified by computing the Jacobian determinant of each MVF and calculating the fraction of pixel locations with negative determinants or folding pixels, which are not physically realistic. The temporal stability in registration predictions is assessed by computing the frame-to-frame vector distances in the MVF and plotting these for each cine sequence in the dataset. These metrics are extracted for every registration and descriptive statistics are reported by patient, treatment site (liver, lung, and pancreas), and for the full cohort. Paired t-tests are used to assess statistical significance of outcomes for each metric. A p-value≤0.05 can be considered statistically significant.

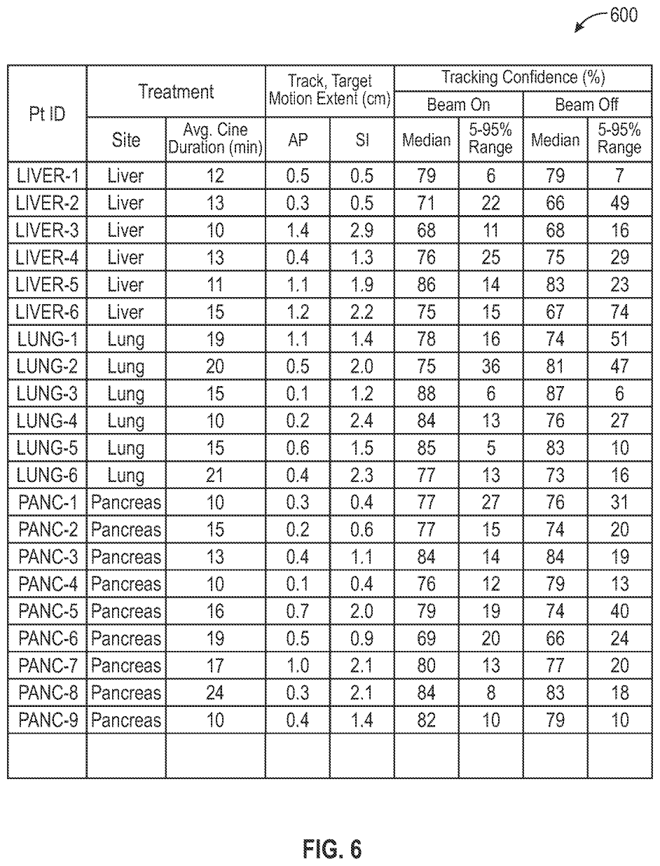

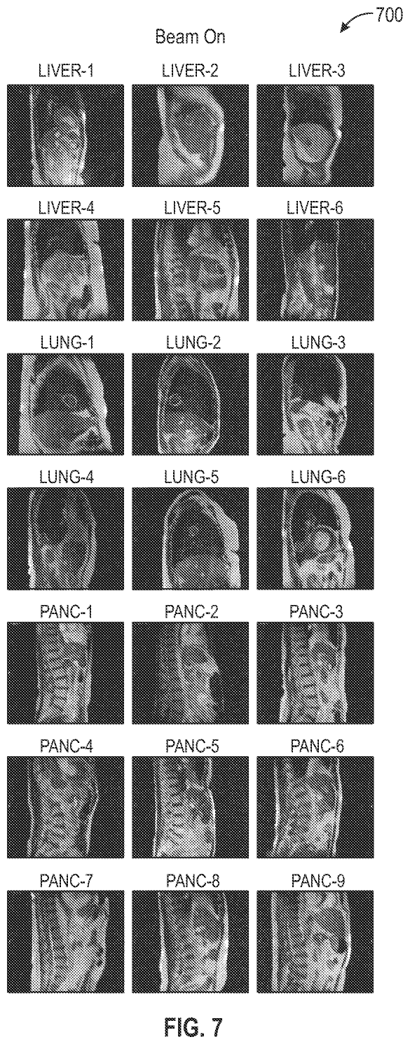

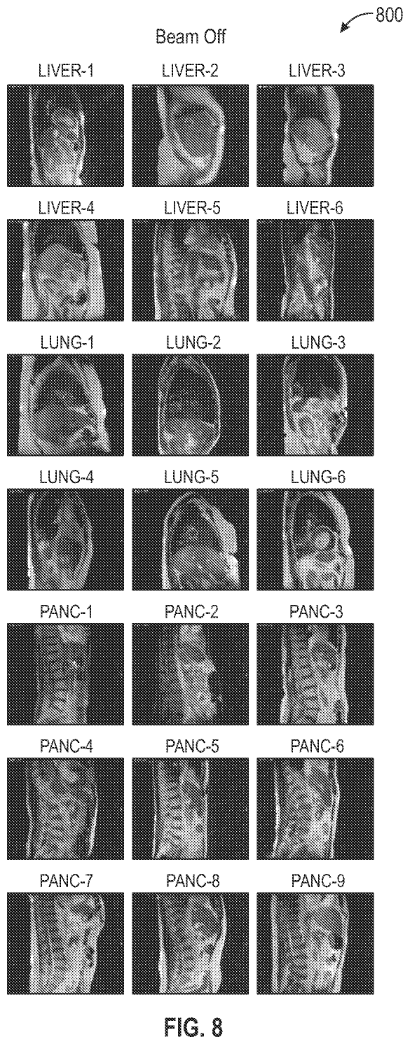

FIG. 6 presents a table 600 showing patient characteristics, cine tracking information, and machine learning fold assignments. AP=anterior posterior, SI=superior inferior, 5-95% denotes the 5th to 95th percentile range. It summarizes cine tracking information extracted from delivery videos for the 21 patients included the analysis. In total, 86 treatment fractions are analyzed. Average treatment cine duration across all patients is 15.2 minutes (7,296 cine frames). Target tracking motion extent from the onboard tracking algorithm ranged from 0.1 to 1.4 cm in the AP direction and 0.4 to 2.9 cm in the SI direction. FIGS. 7 and 8, respectively show exemplary images of cine frames for each patient during beam on (700) and beam off (800) time points. Beam off frames have been sampled from time points corresponding to maximum displacement from the reference position contains example beam on and beam off cine frames demonstrating the motion extent of the tracked tumor target. Median tracking confidence during beam on ranges from 68 to 88% and tracking confidence during beam off ranges from 66 to 87%. Although median tracking confidence is similar across beam on/off time segments for most patients, there is substantial tracking confidence variability from patient to patient as measured using a 5^{th} to 95^{th} percentile range estimation.

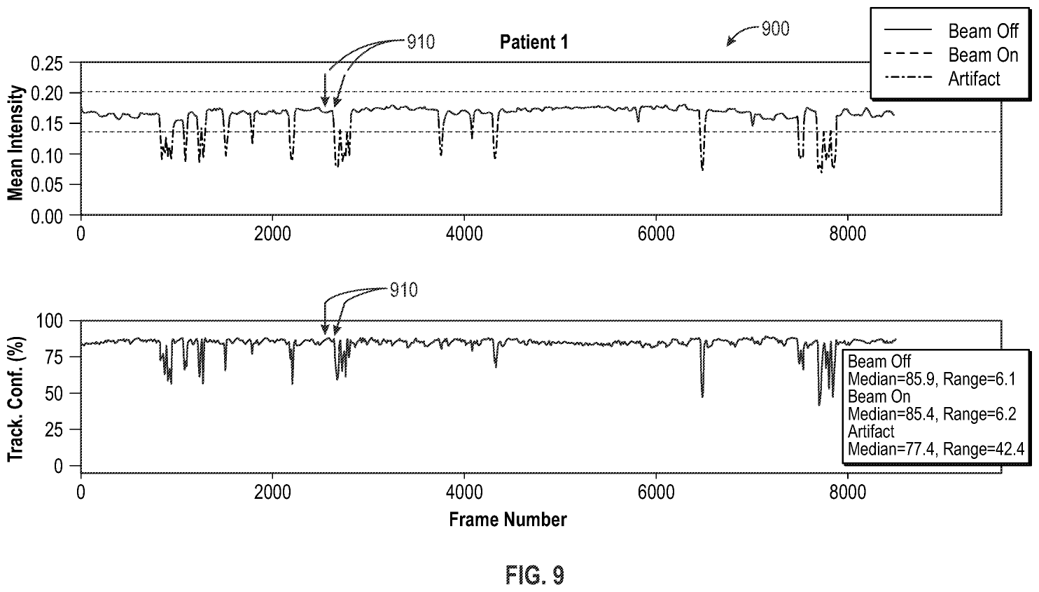

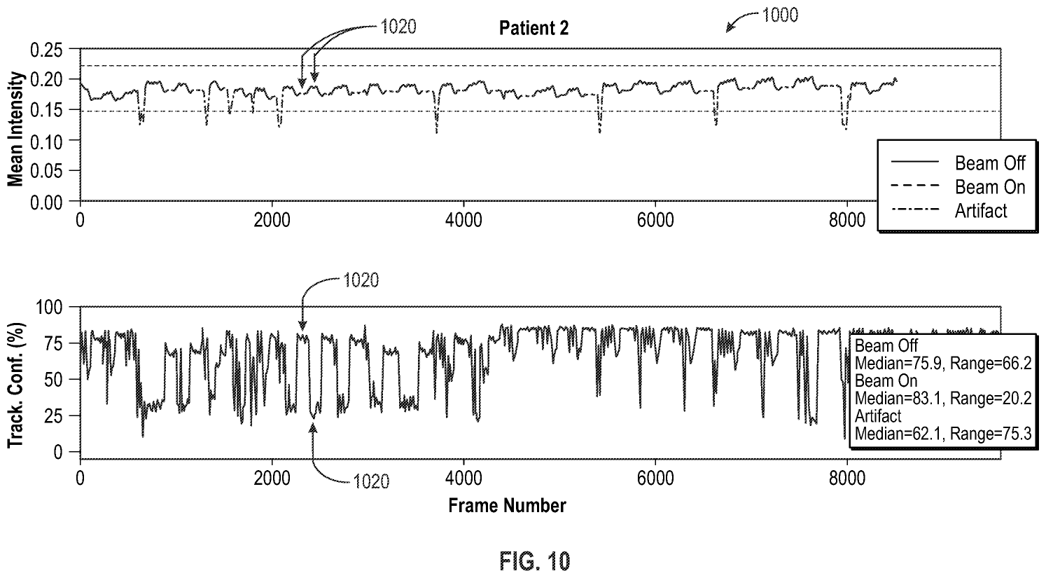

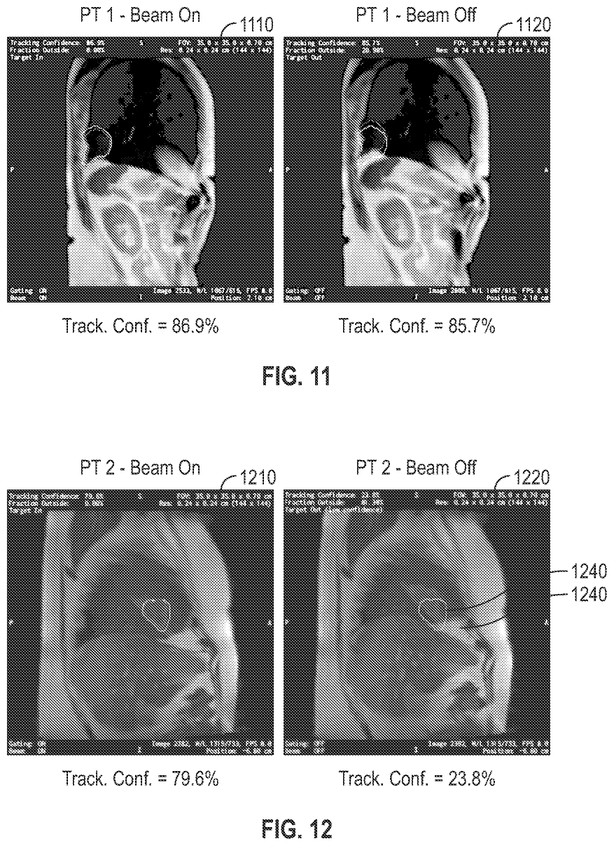

Reference is now made to FIGS. 9-12, which provide a time series extracted and example frames from MR-Linac delivery tracking videos from two patients undergoing MRgRT for lung tumors. FIG. 9 shows a graph 900 of a time series of mean frame intensity and tracking confidence for an exemplary Patient 1. Arrows 910 indicate time points at which example frames are sampled. Dotted lines indicate ±20% thresholds from the median frame intensity used to detect gantry artifacts. Descriptive statistics for tracking confidence during beam on, beam off, and artifact time points are displayed in the legend at right. FIG. 10 is a graph 1010 of time series plots for exemplary Patient 2 exhibiting substantially variation in tracking confidence, with arrows 1020 showing samples. FIG. 11 shows corresponding beam on and beam off image frames 1110 and 1120 for Patient 1. Likewise, FIG. 12 shows corresponding beam on and beam off image frames 1210 and 1220 for Patient 2. Note the significant drop in tracking confidence and misregistration of the tracking contour in the beam off frame 1220 as indicated by the misalignment of the actual anatomical feature (tumor) 1230 versus the identified feature 1240. More particularly, the first cine time series 900 (FIG. 9) exhibits high confidence tracking throughout all time points in the video with the exception of frames affected by gantry artifacts, whereas the second time series 1000 (FIG. 10) exhibits substantial variability in tracking confidence at both beam on and beam off time points. The significant drop in tracking confidence and misregistration of the tracking contour in the beam off frame in the for Patient 2 is clear. The variability in motion extent and tracking confidence indicates that there is a range of difficulty in cine frame registration and target tracking frames from patient to patient. Note that, in the above analysis, the five-fold cross validation by patient is employed for DL model evaluations to compensate for the limited number of patients in the analysis cohort.

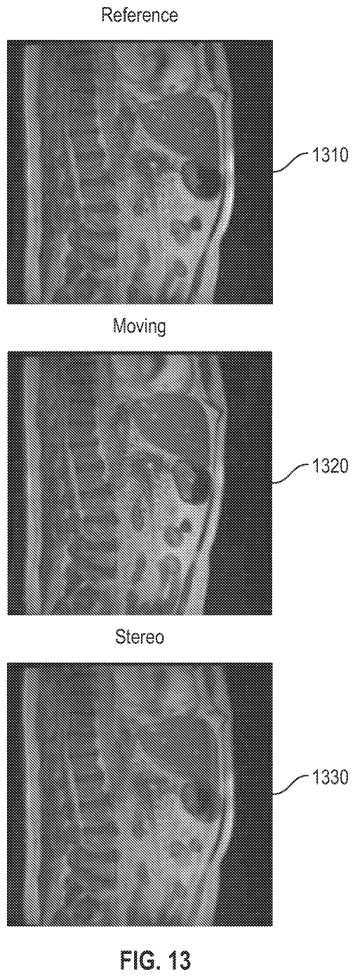

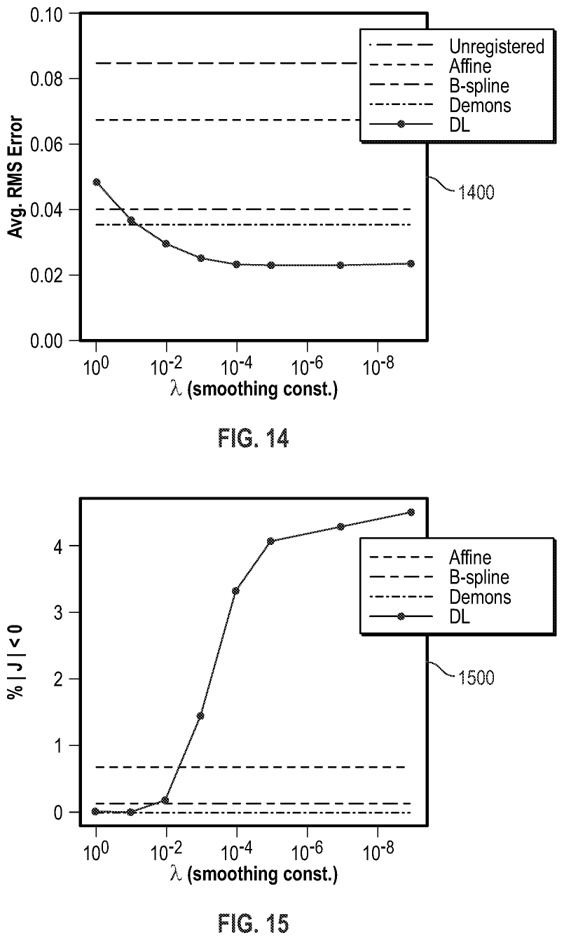

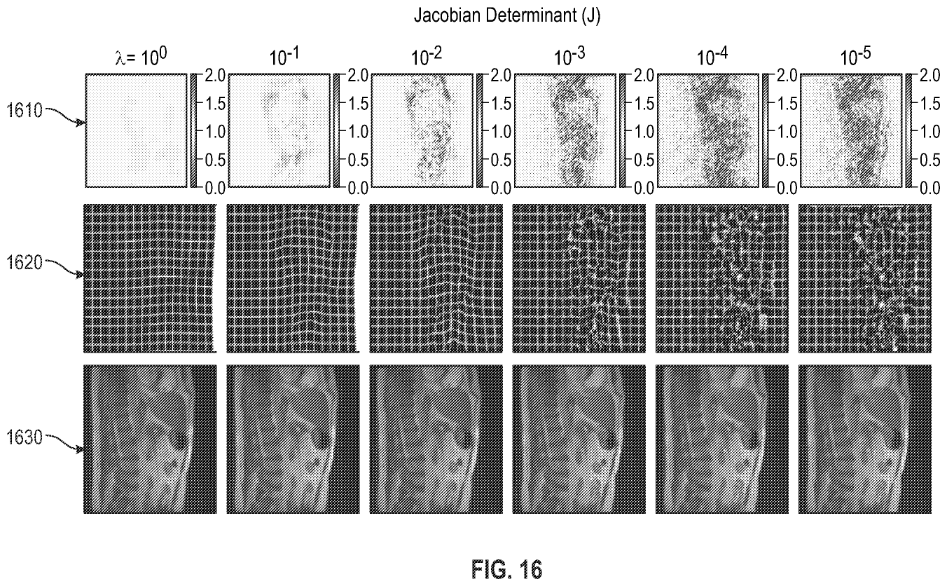

FIGS. 13-16 show a visualization of deformation fields and residual error for various configurations of the DL registration model smoothing hyperparameter (λ), and contains results of DL-based registration error by varying the regularization constant (λ) from 1×10⁰ to 1×10⁻⁸ during model training. As the regularization constant is decreased, the model is less penalized for outputting sharp deformations with larger magnitude. Average RMSE across all test set registrations is lowest for the lowest λ values. However, there is a corresponding increase in folding pixels (negative Jacobians) in the deformation field. It is evident that λ>1×10⁻² results in an unacceptably high rate of folding pixels and a physically unrealistic deformation field. This analysis utilizes outputs from the DL model trained using λ=1×10⁻² for the subsequent assessments. More particularly, FIG. 13 shows an exemplary reference image 1310 and moving image 1320 to be registered, and an associated stereo color visualization 1330 of image misalignment. FIGS. 14 and 15 are respective graphs 1400 and 1500 of mean registration error (RMSE) and fraction of folding pixels (negative Jacobian determinant) on all test set images for four different registration methods. For DL registration, λ can be varied from 1×100 to 1×10-8. FIG. 16 provides a color plot 1610 of Jacobian determinant on example images for each λ, and a corresponding grid plot 1620 of predicted deformation for each λ, and resulting images 1630 from applying DL model registration to the reference image.

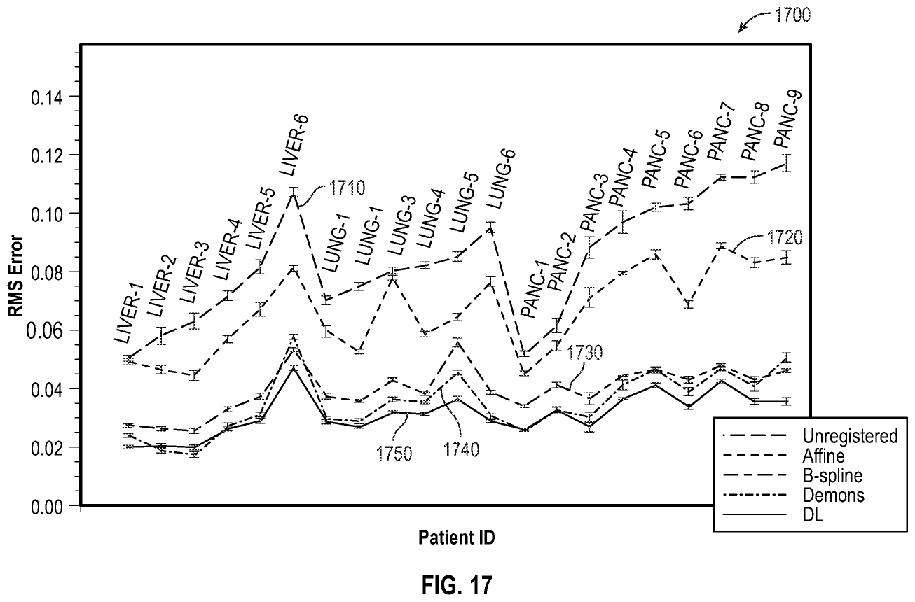

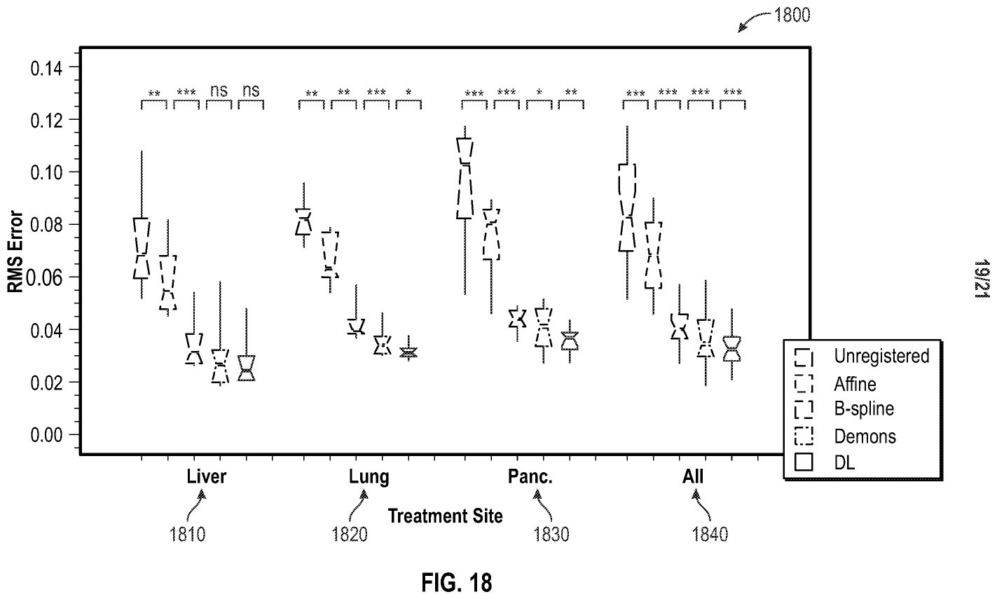

FIGS. 17 and 18 show a comparison of registration error across all patients and treatment sites, and more particularly contains the registration error of DL-based vs conventional registration methods (e.g. affine, b-spline and demons) across all patients and treatment sites, and represents the primary analysis of efficacy of the system and method herein. FIG. 17 shows a graph 1700 of mean registration error for all cine sequences for each patient. Uncertainty bars on the unregistered curve 1710 represent standard error of the mean. More particularly, the graph 1700 contains a line plot of per patient registration error for all 21 patients with uncertainty estimates calculated using the standard error of the mean. The min/max per patient registration errors for affine (curve 1720), b-spline (curve 1730), demons (curve 1740), and the illustrative DL model (curve 1750) are as follows: 0.045/0.089, 0.026/0.059, 0.017/0.059, 0.020/0.048. As clearly indicated per patient registration error is lowest for DL in 90.4% (19 out of 21) patients.

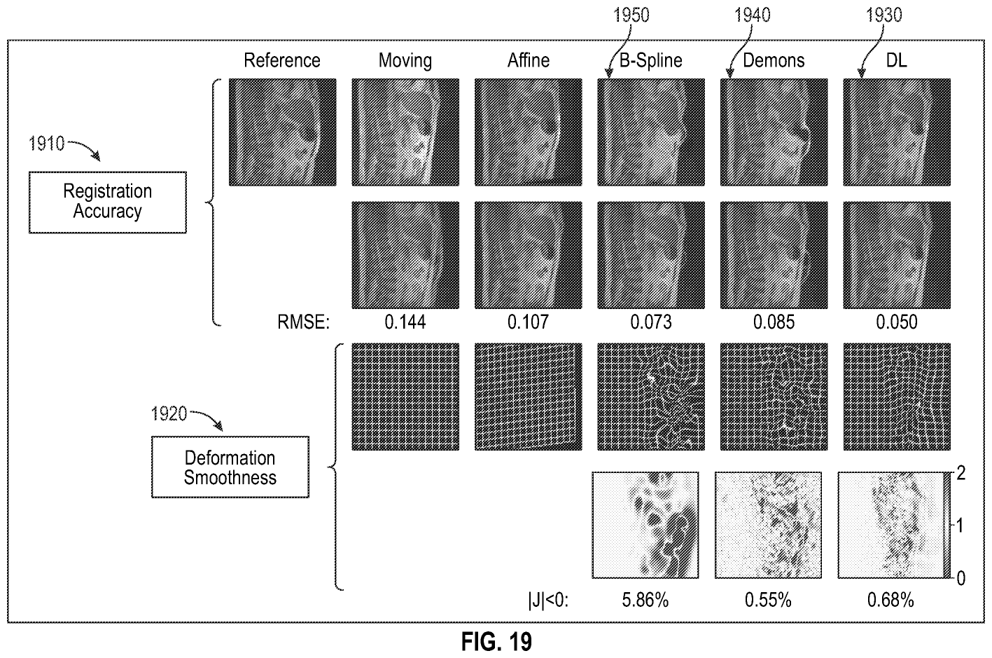

FIG. 18 is a box and whisker distribution plot 1800 of registration error for all cine sequences stratified by treatment site (liver (column 1810), lung (column 1820), and pancreas (column 1830)) as well as for the entire dataset (column 1840). Notched boxes indicate the median and interquartile range with fences indicating the minimum and maximum errors. Statistical comparisons indicate results of a paired t-test comparing the mean registration error for each patient in the category (ns: p>0.05, *: p≤0.05, **: p≤0.01. ***: p≤0.001). As shown, DL-based registration outperforms conventional methods in terms of registration error (shown left-to right in each site as unregistered, and methods: affine, b-spline, demons, DL), and each respective method displaying RMSE: 0.067, 0.040, 0.036, 0.032: paired t-test demons vs DL: t (20)=4.2, p<0.001). Whereas conventional deformable registration is relatively slow (1150 ms and 4583 ms for b-spline and demons, respectively), computation time per frame for DL-based registration is very fast (8 ms per registration), allowing for advantageous use for real-time deployment in MRgRT systems. Reference is made to FIG. 19, which shows comparative registration accuracy/error 1910 and deformation smoothness 1920 metrics for an exemplary cine sequence for each of the 21 patients analyzed. Note temporal consistency of DL-based registration (column 1930) compared to conventional deformable registration from b-spline (column 1950) and demons (column 1940).

The MVF outputs from all three deformable methods have been compared to evaluate their spatial and temporal stability. The fraction of pixels corresponding to misfolding (negative Jacobians) is 0.66%, 0.13%, and 0.19% for b-spline, demons, and DL methods, respectively, indicating slightly better spatial stability for demons compared to the DL model. However, the average change in the MVF over time is significantly lowest for DL-based registration (Euclidean distance b-spline, demons, DL: 1.04, 1.05, 0.77), indicating better temporal stability for the DL model.

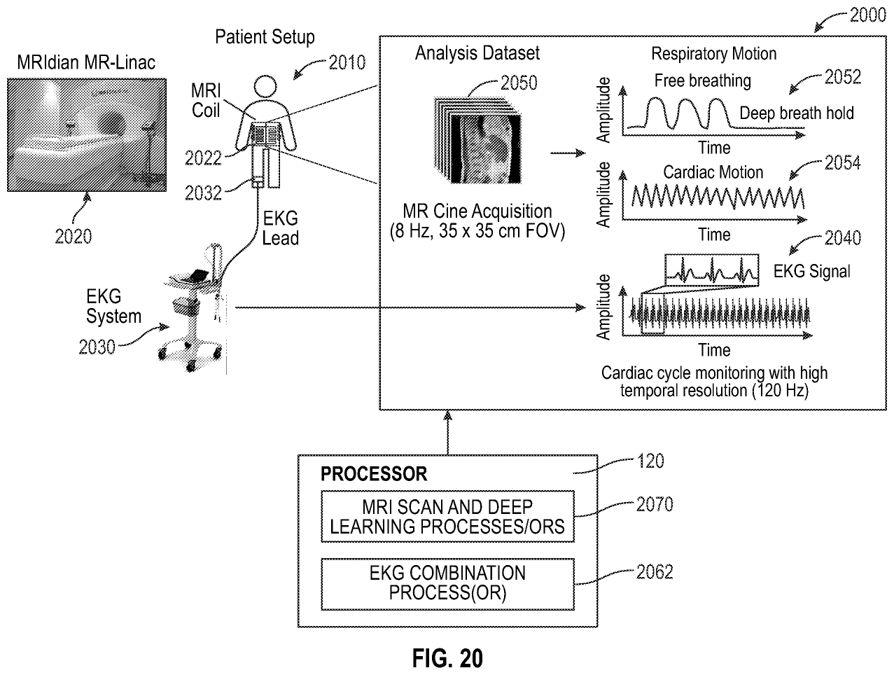

With reference to FIG. 20, and arrangement 2000 is shown in which a patient 2010 wearing an MRI coil 2022 undergoes MR-Linac (2020) therapy. The patient 2010 is also connected to an otherwise conventional electrocardiogram (EKG) system 2030 via (e.g.) a leg cuff 2032, which generates an EKG signal 2040. This can be used in the analysis of the MR Cine images 2050 in conjunction with derived respiratory motion 2052 and cardiac motion 2054. Appropriate interface hardware/software within the process(or) arrangement 120 can be employed to integrate the EKG signal with the MRI scan. As shown, this is represented by a generalized combination/comparison process(or) 2062 that interacts with the scan and DL processes/ors 2070 (similar or identical to processes/ors 122-128 above). Note also that, while applying multiple leads to the patient, including various locations on the chest (e.g. 12 discrete leads), in principle it is possible to provide an adequate EKG signal using one or two leads from the patient's leg as shown. This arrangement thereby avoids potential interference between the leads and MRI scan where such are located near the chest.

### III. Conclusion

When compared with deterministic techniques, including affine, b-spline, and demons image registration algorithms, the above-described DL implementation provides statistically significant reductions in registration error and vastly improved computation time, by a factor of greater than six from affine registration, the next fastest algorithm. While the described system and method relates to motion estimation in the tracking plane, this implementation can be expanded by performing multi-planar registrations for real-time 4D target tracking. By way of background, see van de Lindt, T. N., Nowee, M. E., Janssen, T., Schneider. C., Remeijer, P., van Pelt, V. W. J., Betgen, A., Jansen, E. P. M. & Sonke, J. J. Technical feasibility and clinical evaluation of 4D-MRI guided liver '2 SBRT on the MR-linac. Radiotherapy and Oncology 167, 285-291 (2022). More generally, DL-based image registration can leverage large-scale MR cine datasets to outperform conventional registration methods and is a promising solution for real-time deformable motion estimation in radiation therapy.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments of the apparatus and method of the present invention, what has been described herein is merely illustrative of the application of the principles of the present invention. For example, as used herein, the terms “process” and/or “processor” should be taken broadly to include a variety of electronic hardware and/or software based functions and components (and can alternatively be termed functional “modules” or “elements”). Moreover, a depicted process or processor can be combined with other processes and/or processors or divided into various sub-processes or processors. Such sub-processes and/or sub-processors can be variously combined according to embodiments herein. Likewise, it is expressly contemplated that any function, process and/or processor herein can be implemented using electronic hardware, software consisting of a non-transitory computer-readable medium of program instructions, or a combination of hardware and software. Additionally, as used herein various directional and dispositional terms such as “vertical”, “horizontal”, “up”, “down”, “bottom”, “top”, “side”, “front”, “rear”, “left”, “right”, and the like, are used only as relative conventions and not as absolute directions/dispositions with respect to a fixed coordinate space, such as the acting direction of gravity. Additionally, where the term “substantially” or “approximately” is employed with respect to a given measurement, value or characteristic, it refers to a quantity that is within a normal operating range to achieve desired results, but that includes some variability due to inherent inaccuracy and error within the allowed tolerances of the system (e.g. 1-5 percent). Accordingly, this description is meant to be taken only by way of example, and not to otherwise limit the scope of this invention.

What is claimed is:

1. A system for fast deformable image registration using 2D cine MRI image frames acquired during radiation therapy comprising:
a source of cine MRI images received in approximate real-time from a patient undergoing MRI-guided radiation therapy (MRgRT);
a processor that receives the images and operates a trained deep learning (DL) process that (a) employs a reference image (R) to a moving image as inputs, and outputs a dense motion vector field (MVF) that aligns the images and (b) predicts frame by frame motion from images; and
a guidance process that controls therapy beam operation based upon the prediction.

2. The system as set forth in claim 1, wherein both cardiac and respiratory motion are visible in the images.

3. The system as set forth in claim 2, wherein the DL process extracts, from the images, a predetermined number of hierarchical feature maps at multiple spatial resolutions which are reconstructed into the MVF with spatial resolution equal to the inputs.

4. The system as set forth in claim 3, wherein the MVF is applied by shifting the pixels in the images by associated motion vectors to generate a transformed image (T).

5. The system as set forth in claim 4, wherein the transformed image is generated via a spatial transform.

6. The system as set forth in claim 3, wherein the DL process defines a plurality of network layers and at least one of the network layers is based on a U-Net architecture of a convolutional neural network (CNN).

7. The system as set forth in claim 6, further comprising a loss function with a hyperparameter (λ) defined as:
$${Loss}_{total} = {Loss}_{dissimilarity} + λ {Loss}_{gradients} \qquad ( 1 )$$
where, Loss_{dissimilarity} computes the post-registration mean square error between T and R and Loss_{gradients} computes the magnitude of spatial gradients in the MVF.

8. The system as set forth in claim 6, wherein the network provides network layers in the following order, for each successive layer, of spatial dimensions: 1, ½, ¼, ⅛, 1/16, ⅛, ¼, ½, 1.

9. The system as set forth in claim 8, wherein the loss function is defined by:
$${Loss}_{dissimilarity} = \frac{1}{mn} {\sum }_{i = 1}^{m} {\sum }_{j = 1}^{n} {( {T}_{i , j} - {R}_{i , j} )}^{2} \qquad ( 2 )$$
$${Loss}_{gradients} = \frac{1}{mn} {\sum }_{i = 1}^{m} {\sum }_{j = 1}^{n} {‖ ∇ ( {ϕ}_{i , j} ) ‖}^{2} \qquad ( 3 )$$
where m and n represent the height and width of images.

10. The system as set forth in claim 1, further comprising a combination process that coordinates an EKG signal derived from the patient with the cine MRI images so as to provide data related to cardiac and respiratory motion to the DL process.

11. The system as set forth in claim 10, wherein the EKG signal is derived from one or two legs of the patient.

12. A method for registering using 2D cine MRI image frames acquired during radiation therapy comprising the steps of:
providing a source of cine MRI images received in approximate real-time from a patient undergoing MRI-guided radiation therapy (MRgRT);
applying a trained deep learning (DL) network running on a processor to the images so as to (a) employ a reference image (R) to a moving image as inputs, and outputting a dense motion vector field (MVF) that aligns the images and (b) predicting frame by frame motion from images; and
controlling guidance of a therapy beam operation based upon the predicting

13. The method as set forth in claim 12, further comprising, providing both cardiac and respiratory motion in the images.

14. The method as set forth in claim 13, wherein the step of applying includes extracting, from the images, a predetermined number of hierarchical feature maps at multiple spatial resolutions which are reconstructed into the MVF with spatial resolution equal to the inputs.

15. The method as set forth in claim 14, further comprising, applying the MVF by shifting the pixels in the images by associated motion vectors to generate a transformed image (T).

16. The method as set forth in claim 15, further comprising, generating the transformed image via a spatial transform.

17. The method as set forth in claim 14 wherein the DL network defines a plurality of network layers and at least one of the network layers is based on a U-Net architecture of a convolutional neural network (CNN).

18. The method as set forth in claim 17, further comprising, computing a loss function with a hyperparameter (λ) defined as:
$${Loss}_{total} = {Loss}_{dissimilarity} + λ {Loss}_{gradients} \qquad ( 1 )$$
where, Loss_{dissimilarity} computes the post-registration mean square error between T and R and Loss_{gradients} computes the magnitude of spatial gradients in the MVF.

19. The method as set forth in claim 17, further comprising, organizing the network so that the network layers are arranged in the following order, for each successive layer, of spatial dimensions: 1, ½, ¼, ⅛, 1/16, ⅛, ¼, ½, 1.

20. The method as set forth in claim 19, further comprising, defining the loss function as:
$${Loss}_{dissimilarity} = \frac{1}{mn} {\sum }_{i = 1}^{m} {\sum }_{j = 1}^{n} {( {T}_{i , j} - {R}_{i , j} )}^{2} \qquad ( 2 )$$
$${Loss}_{gradients} = \frac{1}{mn} {\sum }_{i = 1}^{m} {\sum }_{j = 1}^{n} {‖ ∇ ( {ϕ}_{i , j} ) ‖}^{2} \qquad ( 3 )$$
where m and n represent the height and width of images.

21. The method as set forth in claim 12, further comprising, coordinating an EKG signal derived from the patient with the cine MRI images so as to provide data related to cardiac and respiratory motion to the DL network.

22. The method as set forth in claim 21, further comprising, deriving the EKG signal from one or two legs of the patient.
